(19) United States
(12) Patent Application Publication
(10) Pub. No.: US 2006/0206180 A1
(43) Pub. Date: Sep. 14, 2006

(54) Apparatus for non-destructive hyperthermia therapy

(76) Inventor: Luciano Alcidi, Bisenzio (Firenze) (IT)

Correspondence Address: MCGLEW & TUTTLE, PC, P.O. BOX 9227, SCARBOROUGH STATION, SCARBOROUGH, NY, 10510-9227, US

(21) Appl. No.: 10/551,409

(22) PCT Filed: Mar. 22, 2004
(86) PCT No.: PCT/IT04/00141

§ 371 Date: Dec. 20, 2005

(30) Foreign Application Priority Data
Apr. 10, 2003 (IT) FI2003 A 000104

(51) Int. Cl. A61F 2/00

(52) U.S. Cl. 607/102

(57) ABSTRACT

Apparatus for non-destructive hyperthermia therapies, characterized in that it comprises means for generating electromagnetic radio-frequency radiations, connectable to means for the application of said radiations to the human body.

The present invention refers to an apparatus for non-destructive hyperthermia therapies.

A deep, non-destructive hyperthermia, that is, one in which the operating temperature is below 45÷48° C., is known to be usefully applied to various pathologies such as, for example, rheumatoid inflammations, arthritides, tendonitises, etc. However, owing to the thermal characteristics of the skin (which is a good thermal insulator) and to the circulation of liquids below the skin itself (presence of venous and arterial circulation) it is impossible to achieve a significant deep hyperthermia through a simple heat conduction. In fact, in order to raise the internal temperature by a few degrees it would be necessary to increase the surface temperature to such a level that the skin would burn.

Currently, for the treatment of the said pathologies, there are used apparatuses of various type such as, for example, microwaves or ultrasound or laser-operated, or of electroanalgesic type, etc.

The object of the present invention is to provide a new apparatus for hyperthermia therapies able to solve the above problem with efficacy and without damaging the skin.

This result has been achieved, according to the invention, by adopting the idea of making an apparatus having the characteristics indicated in the claim 1. Further characteristics being set forth in the dependent claims.

Among the advantages of the present invention one is that the apparatus results very effective in the treatment of many pathologies, such as rheumatoid inflammations, tendonitises, acute inflammatory forms, etc. In the sports medicine as well, these procedures can be suitably applied in subjects suffering from sprains, muscle strains, contusions, etc.

Another advantage is that the invention allows a drastic reduction of the pain, in relatively short times (in comparison with the known techniques). Moreover, the apparatus is simple to use, easy to operate, and it maintains its characteristics also after prolonged periods of operation.

These and other advantages and characteristics of the invention will be best understood by anyone skilled in the art from a reading of the following description in conjunction with the attached drawings given as a practical exemplification of the invention, but not to be considered in a limitative sense, wherein:

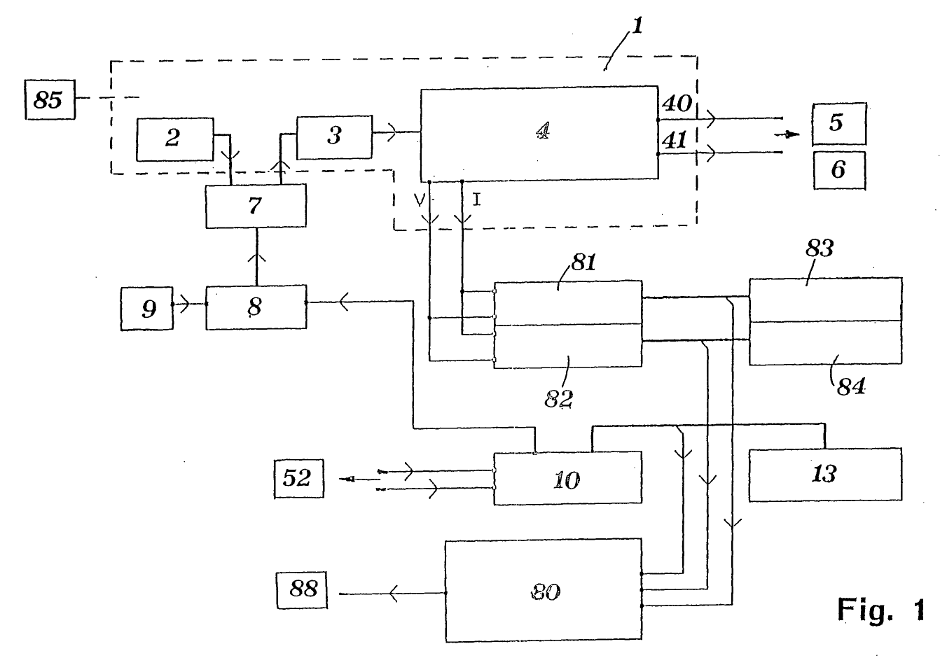

FIG. 1 is a block diagram of a possible embodiment of the apparatus in question;

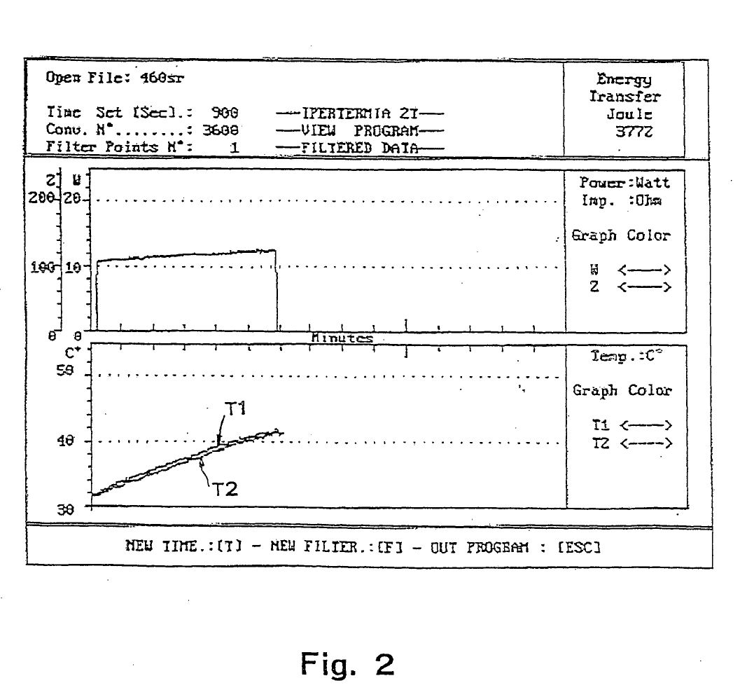

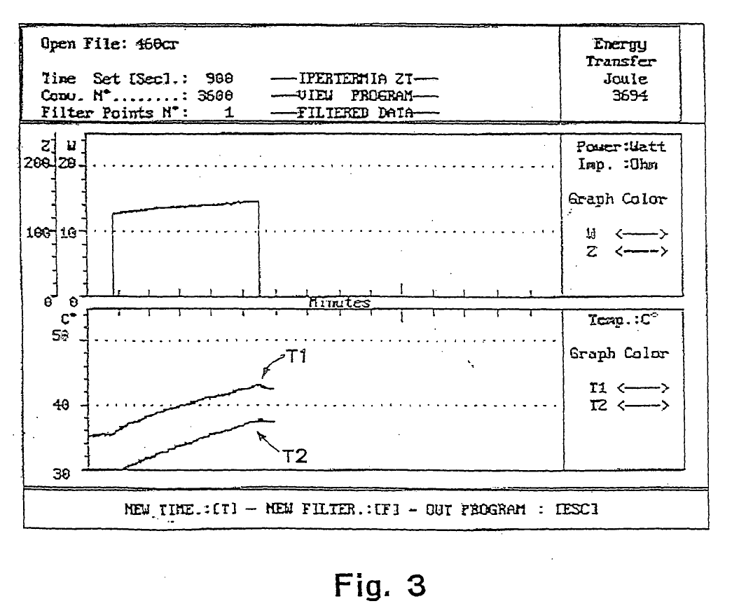

FIGS. 2 and 3 show two graphs relating to the temperature's trend within two sections of the tissue heated up in conformity to the present invention according to two different procedures;

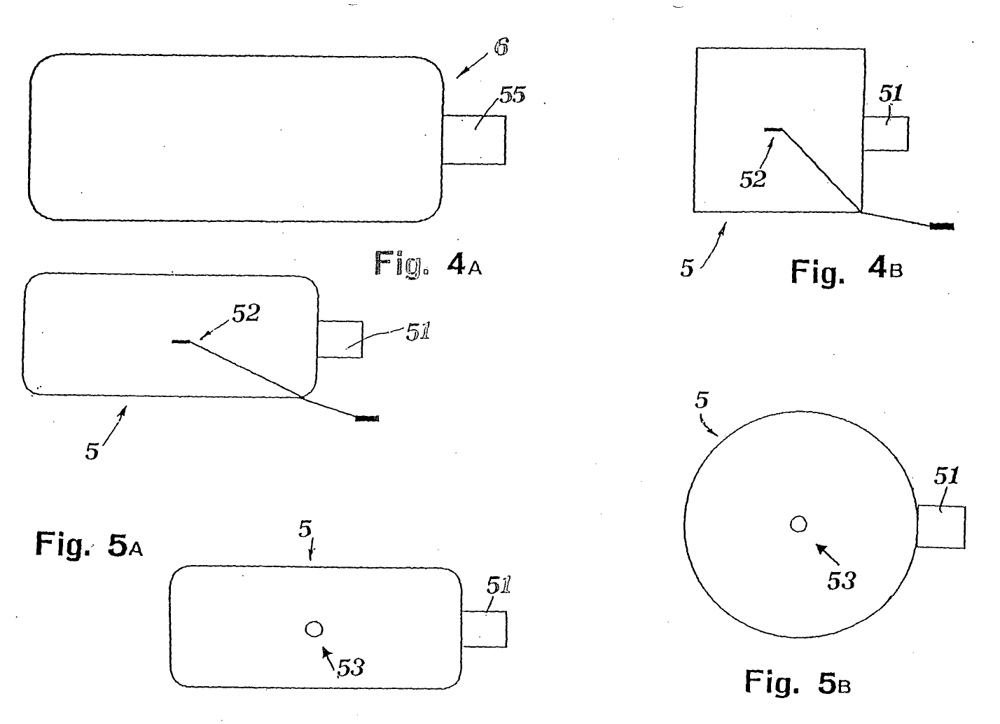

FIGS. 4A and 4B show in plan view from above, an embodiment of two electrodes made according to the invention;

FIGS. 5A and 5B show in plan view from above, a further embodiment of two electrodes made according to the invention;

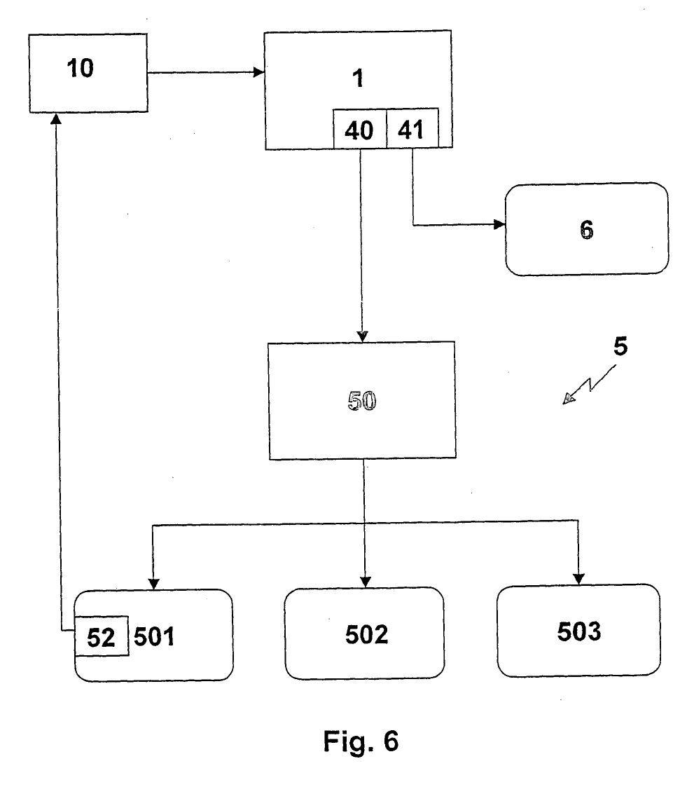

FIG. 6 is a block diagram of a further possible embodiment of the apparatus in question.

An apparatus for non-destructive hyperthermia therapies according to the present invention, is able to transmit energy to the tissues by radio-frequency electromagnetic radiation. The apparatus is able to heat the tissue beneath the skin regardless of the barrier of thermal insulation that the same skin stands up to the heat-conducting processes. In fact, the overheating of the underlying tissue is mainly due to the forces produced by the mid-frequency electromagnetic field, which forces, by interacting with the molecular ions present in the tissue, generate heat.

With reference to the non-limiting block diagram of FIG. 1, the apparatus comprises an RF generator, designated by 1 as a whole. In the example, encircled by a discontinuous line are a plurality of elements making up a generator 1 consisting of: an oscillating circuit 2, a driver 3 and an amplifier 4. On output from the amplifier 4 there are provided two connectors 40 and 41 for connection with corresponding electrodes 5 and 6, to be described later on.

The power of the generator 1 can be adjusted so as to result below a preset threshold; a maximum value may be provided, for example, of 20 W.

Again with reference to the embodiment of FIG. 1, the generator 1 is controlled by a set of circuits which fulfil different functions.

In particular, it is possible: to set the desired temperature reached by the skin; to automatically adjust the output power, so as to keep the temperature of the skin surface at the preset value; to measure the impedance in correspondence of the contact electrodes 5 and 6, the output power and the temperature reached under the delivering electrode; to set the duration of the treatment.

To fulfil the above functions, provision may be made for the following components.

Interposed between the oscillator 2 and driver 3 is a temperature-controlling circuit 7 which controls the power value and is connected to a comparator 8. On input to the comparator 8 are two temperature-related signals, a reference signal programmable by the element designated by the block 9, and a signal detected by a detector circuit 10 connected to relevant sensors 52 (in the example to be described below, being disposed and operating in correspondence of the active electrode 5) which detect the temperature on the skin surface. Associated with the detector circuit 13 is a visualization means, such a display 13, which allows the detected value to be controlled visually. The comparator circuit is connectable to an interface device 80 to be linked, for example, with the gate 88 of a computer to allow the processing of the relevant data.

Also connected to the amplifier 4 are two measuring circuits 81 and 82 intended, respectively, to measure the output power and the impedance in correspondence of the contact electrodes 5 and 6. The two measuring circuits 81 and 82 can be connected, as shown in FIG. 1, to relevant displays 83 and 84 for the visualization of the detected values.

Moreover, connected to the RF-radiations generator 1, is a timer circuit 85 able to set the treatment duration.

As above mentioned, on output from the generator 1, more specifically, downstream of the amplifier 4, there are provided two electrodes 5 and 6. In particular, as illustrated in the example of FIGS. 4A-5B, the electrodes are made up of a first contact plate 6 (shown only in FIG. 4A) which represents the reference electrode 6, and of a second plate 5, of smaller dimensions and provided with a thermocouple, which represents the active electrode 5. By way of example, the first plate 6 may exhibit a surface area of about 80 cm² and be provided with a connector 55 associable with the connection 41 provided on the generator 1.

Both the reference electrode 6 and active electrode 5 are to be considered of disposable type or, at the most, reusable on subsequent applications for the same patient, and they can be made through the same technology.

The active electrode 5 may be, as already mentioned, either of disposable or reusable type; it being preferably made up of a conductive membrane having suitable dimensions. For example, the active electrode may exhibit an area extending over (10×10) cm by using larger reference plates, for example of (15×15) cm. The active electrode is provided with a connector 51 suitable for linking the RF generator 1 via the connection 40, for example. The membrane of the active electrode 5 is coated with a layer of adhesive and conductive gel able to ensure a proper and full contact with the patient's skin. The electrode, moreover, is provided with one or more temperature sensors such as, for example, one or more thermocouples 52. This makes it possible, as above indicated, to monitor in real and continuous time the temperature reached by the skin itself; besides, the value detected by the thermocouple 52 also allows continuously adjusting the delivery of RF power, so as to keep the surface temperature at a steady value approximately matching that of the preset threshold.

The structure of the active electrode 5 shall have shapes and dimensions suited for the region of the body to be treated. The shaping will be therefore rectangular, square, rounded, circular, etc.

The temperature sensors 52 may be incorporated in the same electrode (such as in the examples of FIGS. 4A and 4B) so as to result themselves disposable when the electrode is of this type. Alternatively, as shown in the examples of FIGS. 5A and 5B, the electrodes may exhibit a seat 53 complementarily matchable with a corresponding connector of the sensor.

Should the regions interested by the pathology be of significant extension, an embodiment of the invention would be used able to distribute the RF sequentially over more active electrodes, the reference electrode being the same, so as to avoid the delivery of high power as necessary when using larger electrodes.

An example of such embodiment is diagrammatically illustrated in FIG. 6 wherein the components similar to those of FIG. 1 are given the same reference numbers, numeral 5 indicating the complex of elements that define the active electrode. The various active electrodes (which, in the non-limiting example, are in number of three and designated by 501, 502 and 503), all of them having the same dimensions, could be so disposed as to cover the interested region and connected with a switching system 50 linking them sequentially to the RF source composed of the generator 1. The element 50 could be made up of a sequential switch connected to the output 40 of the RF generator 1 and to the three active electrodes 501, 502 and 503.

This will make possible to heat a larger area with the same power although, obviously, with longer application times. The control temperature can be measured on only one (in FIG. 6 designated by 501) of the active electrodes being used, with provision of maintaining this electrode connected for a time a little longer than for the others, in order to make sure that the reading of the reference temperature results higher than that of the others.

During the experimentations being carried out, it has been found that, in order to provide a proper heating of the tissue, the specific recommended power to be applied (measured in Watt/cm²) is in the order of 0.3 W/cm².

Moreover, during the experimentation, the presence of the interface 80, especially of analog/digital type, has shown to be very useful, although the instrument is able to operate autonomously as well. To achieve a standardization of the application procedures for the individual pathologies during the experimentation, and for a correct filing upon a routine usage as well, use is made of the analog/digital interface 80 allowing a direct connection, via a serial gate 88, with a computer which, when provided with a dedicated software, is able to register all those parameters such as output power, operating impedance and skin temperature.

During the experimentation, the preset apparatus was used for applications on subjects affected by acute inflammatory forms and suffering from pains in, respectively, a knee, first phalanx of the thumb with a swelling at its joint, and in the lumbar position of the back.

In all these cases, the application time was set on 25 minutes. After the first application, each patient experienced a significant reduction of the pain.

The applications were repeated at regular intervals of 24 hours and, upon the fourth application, all the patients had their pains completely relieved and, in addition, the swelling at the joint between the metacarpus and the first phalanx of the thumb virtually disappeared.

From this approach in the treatment it seems that the non-destructive hyperthermia produced from a radio-frequency electromagnetic radiation is able to immediately raise the pain's threshold of the local receptors (attenuation or disappearance of the pain upon completion of an individual application) and to have a significant anti-inflammatory effect.

FIGS. 2 and 3 refer to an in vitro experimentation and provide some values relevant to parameters of the instrument used for the measurements. These figures point out that the heating of the tissue, as obtained from the radio-frequency electromagnetic radiation produced by the said instrument, is able to overcome the thermal barrier opposed by the skin. In fact, the graph of FIG. 2 shows the surface temperature and the temperature at 2 cm deep in the tissue—with a uniform starting temperature—designated, respectively, by the references T2 and T1. In this case, as illustrated by the graph, the two temperatures can be considered as overlapping. Plotted in the upper part of the graph is the output W power-versus-time curve.

FIG. 3, instead, show the trend of the same temperatures within the tissue when the starting temperature is not uniform; in fact, in this case the in-depth temperature T1 is maintained by a heat source, other than the said instrument, at a value which is 7° C. higher than the surface-relating T2. As can be seen in the graph, such ΔT is maintained throughout the measurement. This shows how it is possible to achieve a deep hyperthermia by a radio-frequency electromagnetic radiation also in the presence of the thermal insulation provided by the skin.

Practically, the construction details may vary in any equivalent way as far as the shape, dimensions, elements disposition, nature of the used materials are concerned, without nevertheless departing from the scope of the adopted solution idea and, thereby, remaining within the limits of the protection granted to the present patent.

1. Apparatus for non-destructive hyperthermia therapies, characterized in that it comprises means for generating radio-frequency electromagnetic radiations, connectable to means for the application of said radiations to the human body.

2. Apparatus according to claim 1, characterized in that the said means comprise an active electrode and a reference electrodes, the said active electrode being provided with means for the detection of the skin's temperature.

3. Apparatus according to claim 2, characterized in that the said means for the detection of the skin's temperature are made up of at least a sensor incorporated in the electrode.

4. Apparatus according to claim 2, characterized in that the said means for the detection of the skin's temperature are made up of at least a sensor which can be connected to the apparatus and removably associated with the active electrode in correspondence of a relevant seat thereof.

5. Apparatus according to claim 2, characterized in that the said means for the detection of the skin's temperature are connected to a control circuit connectable to and acting on said means for generating radio-frequency radiations.

6. Apparatus according to claim 2, characterized in that the said electrodes consist of conductive plates or membranes.

7. Apparatus according to claim 2, characterized in that the structure of the active electrode is complementary shaped with respect to the body's region of the patient to be treated.

8. Apparatus according to claim 2, characterized in that the said reference electrode has dimensions larger than those of the active electrode.

9. Apparatus according to claim 2, characterized in that it comprises more active electrodes connected to a switch device able to connect in sequence said active electrodes to said means for generating radio-frequency radiations.

10. Apparatus according to claim 1, characterized in that it comprises means for adjusting the temperature reached on the skin and able to vary the output power in order to keep the skin's temperature at a preset value.

11. Apparatus according to claim 1, characterized in that it comprises means for measuring the output power and the impedance in correspondence of the application means.

12. Apparatus according to claim 1, characterized in that it comprises means to preset the duration of the treatment.

13. Apparatus according to claim 1, characterized in that it comprises means for connection with an electronic processor.
